(19) United States
(12) Patent Application Publication
(10) Pub. No.: US 2015/0065694 A1
(43) Pub. Date: Mar. 5, 2015

(54) Preassembled hybrid nanocluster plasmonic resonator for immunological detection and serotyping of virus and microbes

(71) Applicants: Fanqing Frank Chen, Moraga, CA (US); Mohamed Shehata Draz, Tanta (EG)

(72) Inventors: Fanqing Frank Chen, Moraga, CA (US); Mohamed Shehata Draz, Tanta (EG)

(73) Assignee: United States Department of Energy, Washington, DC (US)

(21) Appl. No.: 14/472,516

(22) Filed: Aug. 29, 2014

Related U.S. Application Data
(60) Provisional application No. 61/872,096, filed on Aug. 30, 2013.

(51) Int. Cl. G01N 33/569; C07K 17/14

(52) U.S. Cl. CPC G01N 33/56994; G01N 33/56983; C07K 17/14; Y10S 977/774; Y10S 977/81; B82Y 15/00
USPC 530/391.3; 977/774; 977/810

(57) ABSTRACT

Here, we describe a preassembled plasmonic resonance nanocluster. One embodiment is used for microbe detection and typing. The metallic nanoparticle acceptors with microbe surface antigen epitope, and quantum dot (QD) donors with Fab antibody, are assembled into an immuno-mediated 3D-oriented complex with enhanced energy transfer and fluorescence quenching. The coherent plasmonic resonance between the metal and QD nanoparticles is exploited to achieve improved donor-acceptor resonance within the nanocluster, which in the presence of microbial particles is disassembled in a highly specific manner. The nanocluster provides high detection specificity and sensitivity of the microbes, with a sensitivity limit down to 1-100 particles per microliter and to attomolar levels of a surface antigen epitope. A few specific examples of the plasmonic resonance nanocluster used in microbe detection are disclosed along with ways in which the complex can be easily modified for additional microbes.

### PRIORITY

The present patent application claims priority to the corresponding provisional patent application Ser. No. 61/872,096, entitled “Preassembled Hybrid Nanocluster Plasmonic Resonator for Immunological Detection and Genotyping of Virus and Microbes,” filed on Aug. 30, 2013.

### GOVERNMENT RIGHTS

The United States Government has rights in this invention pursuant to DOE Contract No. DE-AC02-05CH11231 between the U.S. Department of Energy and the University of California, as operator of Lawrence Berkeley National Laboratory.

### FIELD OF THE INVENTION

The present invention relates to plasmonic resonance quenching in microbe detection.

### BACKGROUND

Recent advances in nanotechnology present exciting opportunities to create interclustering hybrid nanostructures with broadly tunable and enhanced properties. Successful use of noble metals, especially gold, has inspired a great body of research efforts on photonic nanostructures. Gold nanoparticles (AuNPs) with characteristic surface plasmon resonance (SPR) were reported to drive intrinsic emission enhancement or photoquenching effects when interacting with photon emitters in the proximity. These nanoscale distance-based plasmonic effects have guided increasing development for the integration of AuNPs into various nanoscale structures. On the other hand, the inherent optical properties of quantum dots (QDs), such as high quantum yield, size- and composition-tunable emission, broad excitation range, narrow and symmetric emission spectra, and excellent photostability, have enabled them to be ideal companions for AuNPs in fabrication of potent hybrid photonic nanostructures. A plasmon-mediated nanomaterial surface energy transfer (NSET) mechanism analogous to fluorescence resonance energy transfer (FRET) was described in such hybrid nanostructures. Au-QD as a pair of oscillating dipoles undergoes long-range dipole-dipole coupling, and the excitonic energy of QDs is known to be resonantly funneled and quenched into the plasmon of AuNPs. Following the concept of FRET, the energy transfer between QDs and AuNPs can be expressed in eq 1, which relates the resonance energy transfer efficiency E, to the Förster distance R₀, the Au-QD interdistance r, and the n number of Au-acceptors interacting with a single QD-donor; where the Förster distance is the distance at which the energy transfer is equal to 50%.

$$E = \frac{{R}_{0}^{6}}{{nR}_{0}^{6} + {r}^{6}} \qquad ( 1 )$$

The excited surface plasmons Eₛ are dependent on the strength of the applied electrical field E₀, and the induced (dipole) field in the particle. For a spherical particle with radius rₘ, and dielectric constant ε, placed in a layer with dielectric constant ε₁, the overall field gain (G) of plasmonics can be represented as

$$\begin{matrix}G ( ω ) = \frac{{E}_{s} ( ω )}{{E}_{0} ( ω )} \\ \approx 1 + \frac{ɛ ( ω ) - {ɛ}_{1}}{ɛ ( ω ) + 2 {ɛ}_{1}} {( \frac{{r}_{m}}{r + {r}_{m}} )}^{3}\end{matrix} \qquad ( 2 )$$

AuNPs surface plasmons were documented to attain energy-transfer distance up to 100 nm and by coupling with the QD-excitons this achieved enhancements beyond the limitations of traditional FRET. We postulate that improvement and optimization of Au-QD interclustering and spatial arrangement will result in augmented plasmonic interaction, affording enhanced energy transfer with a minimal background interference, which will in turn significantly broaden the potential applications of hybrid Au-QD nanoplasmonics. The key is to control the interclustering parameters such as internanoparticle distance, spectral overlap, spatial orientation, and acceptor-donor ratio, so as to permit an efficient energy transfer without affecting the achievable sensitivity to perturbation of plasmonic resonance. Several studies have successfully suggested that Au-QD hybrid clusters are superior building blocks for plasmonic sensing nanophotonics. So far, fluorescence quenching-based schemes have been established for the detection of heavy metal ions, enzyme activity, blood glucose level, DNA, protein glycosylation, and more recently for the detection of prion protein. Yet no previous effort has been successful in applying plasmonic resonance quenching to microbe detection. Virus and bacteria are orders of magnitude larger than small molecules and macromolecule complexes; their large sizes impact directly the distance-dependent resonance mechanism of NSET, leading to poor plasmonic resonance yield and imposing a challenge to an efficient detection of microbes. Design embodiments presented here allow the large size of microbes to be detected through plasmonic resonance with NSET.

### BRIEF SUMMARY

One preferred embodiment is directed toward a nanocluster plasmonic resonator complex and a process for using the complex for efficient detection of microbes. Briefly, the nanocluster plasmonic resonator complex is a nanocluster composed of multiple capping nanoparticle conjugates and a single core nanoparticle conjugate. The peptide epitope and the Fab fragments targeting the epitope have been used to mediate the preassembly of the Cap-Core nanocluster. The synthetic peptide is conjugated to metallic nanoparticles to prepare the nanocluster capping conjugates while the Fab fragments were covalently linked to amine-derivatized, polyethylene glycol-coated (PEG-coated) QDs to prepare the nanocluster core conjugates.

One or more embodiments of the invention relate to a nanocluster complex for Hepatitis B virus (HBV) sensing composed of multiple capping nanoparticle conjugates and a single core nanoparticle conjugate. The peptide epitope, corresponding in sequence to HBV surface antigen preS2 was conjugated to AuNPs to prepare the nanocluster core conjugates.

The multiple embodiments of the present invention described herein have many advantages, including but not limited to those described above. However, the invention does not require that all advantages and aspects be incorporated into every embodiment of the present invention.

### BRIEF DESCRIPTION OF THE DRAWINGS

These and other features, aspects, and advantages of the present invention will become better understood with reference to the following description, appended claims, and accompanied drawings where:

FIG. 1 is a schematic diagram of one embodiment of the nanocluster plasmonic resonant complex.

FIG. 2 is a schematic diagram of one embodiment of the process to create the complex.

FIG. 3 is comprised of data containing graphs showing nanocluster caps preparation and characterization.

FIG. 4 is comprised of data containing graphs showing validation of monofunctionality of AuNP and nanocluster caps structure with surface peptide epitope.

FIG. 5 shows nanocluster core preparation and characterization.

FIG. 6 shows the nanocluster complex assembly.

FIG. 7 shows the nanocluster sensing action of one embodiment of the invention.

FIG. 8 shows the nanocluster complex interaction kinetics.

FIG. 9 Schematic depicts the theoretical NPR inter-cluster distance.

FIG. 10 TEM characterization of nanocluster complex with formation of distinctive clusters without large aggregations.

### DETAILED DESCRIPTION

One or more embodiments of the present invention are directed to a nanocluster complex comprised of a preassembled plasmonic resonance nanocluster.

One preferred embodiment of the invention is a nanocluster complex for identifying the presence of one or more microbes comprising: (1) a single core nanoparticle conjugate (CNC), wherein said CNC comprises a quantum dot (QD) covalently linked to one or more fragment antigen-binding (Fab) antibodies; (2) one or more multiple capping nanoparticle conjugates (MCNCs), wherein each said MCNC comprises a metal nanoparticle functionalized with one or more peptide epitopes; and (3) at least one linkage between said CNC and one or more MCNCs wherein said linkage comprises one or more peptide epitopes of said MCNCs and said Fab antibodies targeting said epitopes on said CNC.

One or more embodiments of the invention are a preassembled nanocluster plasmonic resonator complex that contains multiple epitopes allowing for high-throughput serotyping as well as measuring pathogens.

One or more embodiments comprise a novel nanocluster plasmonic resonator complex for detection of viruses and bacteria with high specificity and sensitivity. The preassembled nanocluster plasmonic resonator complex is comprised of multiple capping nanoparticle conjugates and a single core nanoparticle conjugate. The peptide epitope and the Fab fragments that target the epitope mediate the preassembly of the nanocluster plasmonic resonator complex in assembling the cap-core nanocluster. The capping synthetic peptide is conjugated to a metal nanoparticle while the Fab fragments are covalently linked to amine-derivatized, polyethylene glycol-coated quantum dots to prepare the nanocluster core conjugates. Multiple embodiments of the invention are described, including examples. These examples are not meant to limit, rather to show the invention in a select few of the embodiments. These examples include, but are not limited to, detection of HBV, HCV, and EBV using the described preassembled nanocluster plasmonic resonator complex. By using the techniques described herein, modification of the Fab fragments will also allow for the measuring of multiple epitopes. Generating Fab fragments from monoclonal antibodies of pathogen surface antigens will allow for the nanocluster plasmonic resonator complex to detect a multitude of viruses, microbes, and bacteria in a multiplex diagnostic assay.

### Single Core Nanoparticle Conjugate (CNC)

Each CNC comprises a quantum dot (QD) and at least one fragment antigen-binding antibodies (Fab) through a linkage. In a preferred embodiment, the Fabs are generated from antibodies against a peptide epitope. IgG molecules are digested with immobilized ficin in the presence of cysteine. Fabs are separated from the intact Fc fragments and undigested IgG, and recovered by protein A column chromatography. The eluted Fab fractions are washed out by PBS and purified by centrifugation. Fractions containing the purified Fab fragments are pooled and the protein concentration determined. There are well-known methods of generating Fabs in the art, one of which is exemplified in Krell et al. PCT/EP2012/068532 filed on Sep. 20, 2012 and is herein incorporated in its entirety by reference.

To create the quantum dot-Fab core conjugate, a fluorescent quantum dot amine-derivatized, PEG-coated nanocrystals solution was activated with a heterobifunctional crosslinker yielding a maleimide-modified particle. In each reaction, Fabs were simultaneously reduced by incubating in dithiothreitol (DTT) in saline. Excess crosslinker and DTT are removed using desalting columns. Activated fluorescent QDs are then incubated with reduced Fab fragments and the reaction quenched. The QDs and Fab fragment solution is concentrated via ultrafiltration. The concentrated solution is purified from the uncoupled free Fabs in a separation media column. The final QD-Fab conjugate is collected and stored. The conjugation is then assessed through procedures involving characterization of the bioconjugates by optical characterizations and the gel electrophoresis technique.

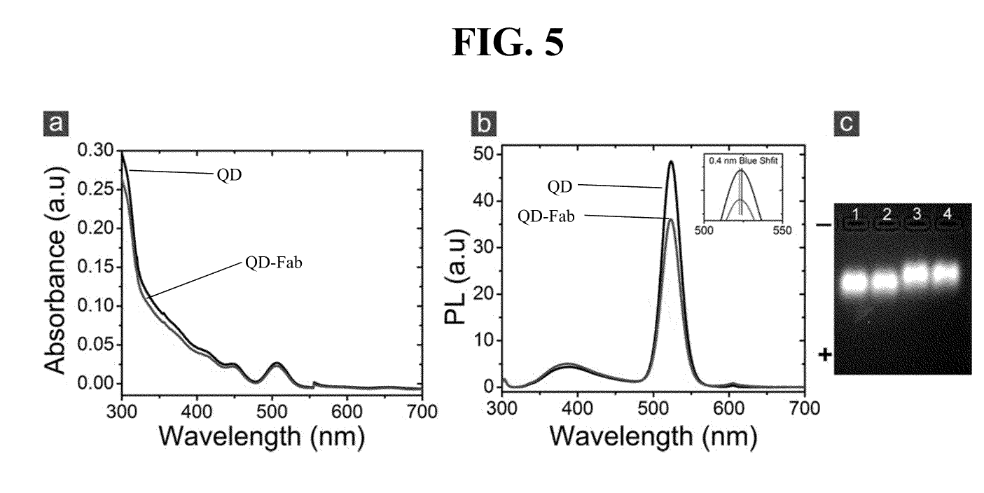

The Fabs targeting the peptide epitope are used to mediate the preassembly of the Cap-Core nanocluster. FIG. 5 shows the optical characterization of the Core through UV-vis absorption spectra and photoluminescence spectra of QDs and QD-Fab core revealing almost identical profiles before and after conjugation with a slight shift in for photoluminescence spectrum. Panel C of FIG. 5 shows the gel electrophoresis of QDs (lanes 1 and 2) and QD-Fab conjugates, where the shift in band size shows the successful conjugation of the Fab fragment with the QD.

### Multiple Nanocluster Capping Conjugates

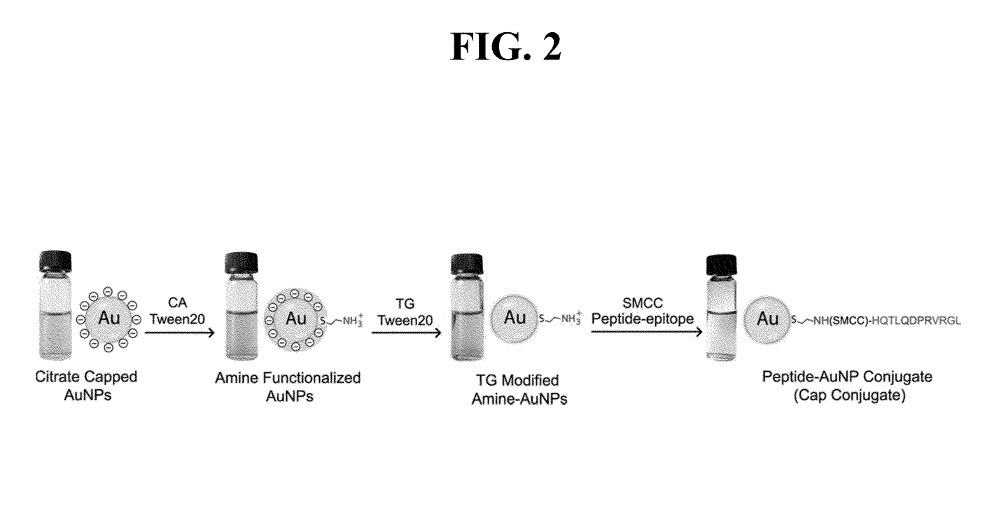

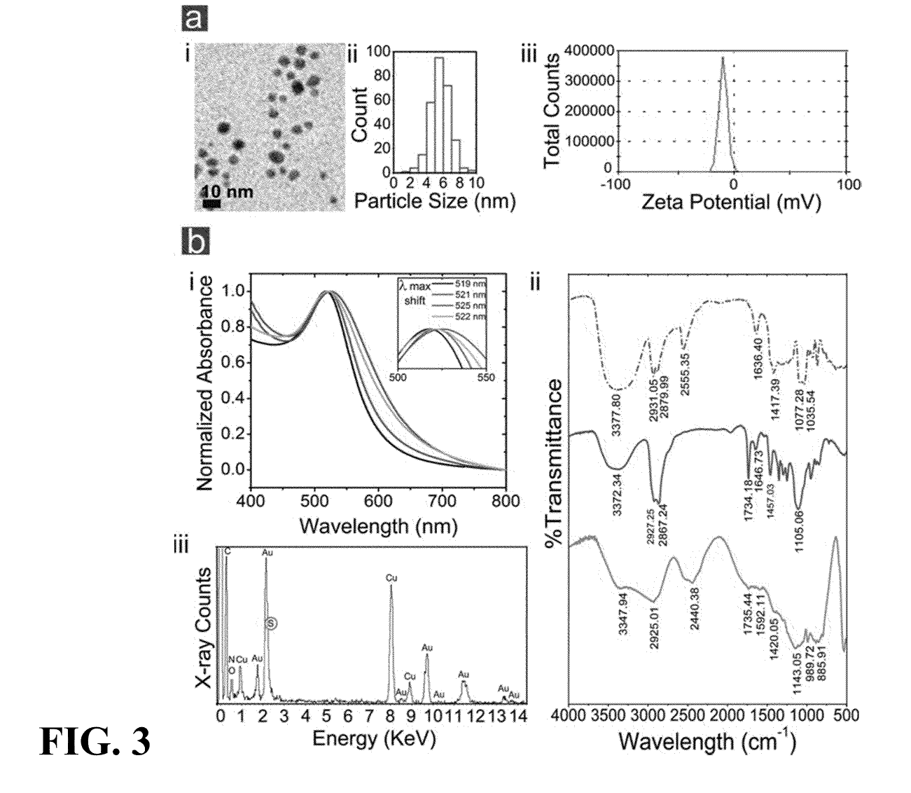

Embodiments of the invention comprise nanocluster capping conjugates. In a preferred embodiment, these nanocluster capping conjugates are spherical in shape with an average diameter of 4-7 nm, shown in FIG. 3. As FIG. 2 shows, metallic nanoparticles are used for the preparation of monofunctionalized capping conjugates by coupling with the targeted peptide epitope. The metallic nanoparticles are stabilized with negative ions and then modified using an aminothiol in the presence of a detergent. A glycerol is then added. A crosslink reagent is then used to functionalize the metallic nanoparticles through interactions with the amino group on the particle surface. In order to ensure a proper linkage, a peptide epitope is modified with a C-terminal cysteine. That modified peptide epitope couples through the terminal cysteine thiol group to the active maleimide group introduced to the metallic nanoparticles by the crosslink reagent.

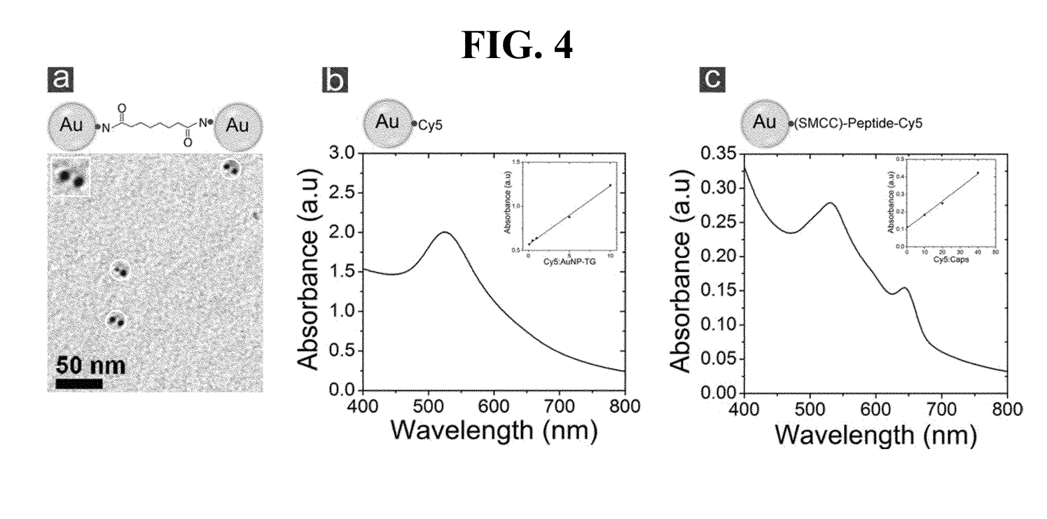

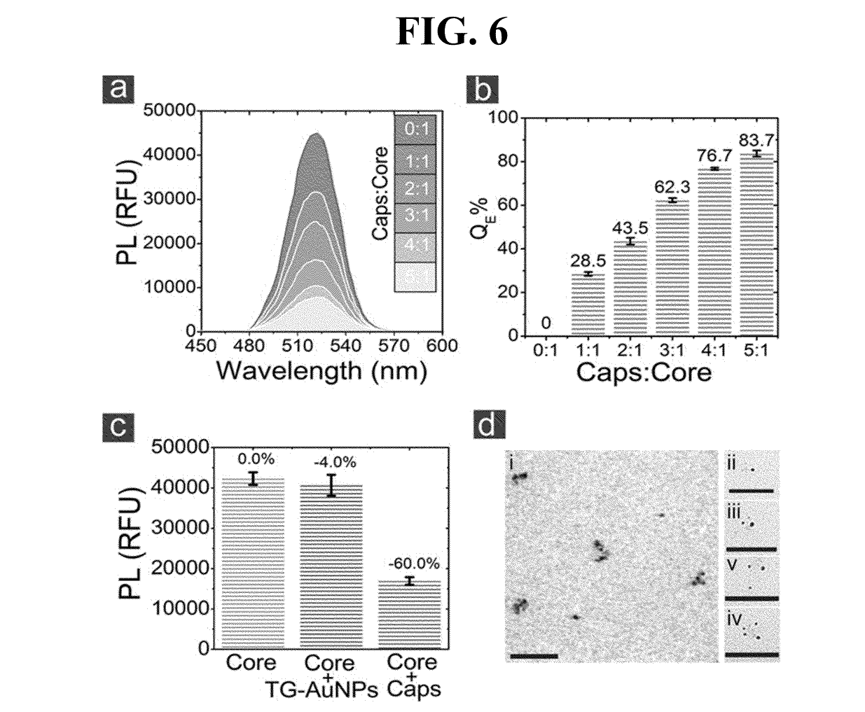

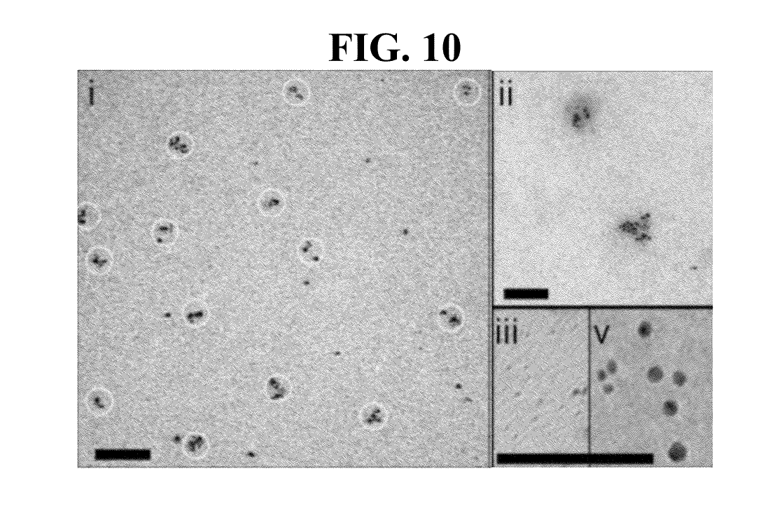

This preparation of the monofunctionalized capping conjugates with a single peptide epitope is a key factor in the quenching efficiency. The quenching efficiency is balanced on the monofunctionalized capping conjugates with a single peptide epitope. By increasing the number of monofunctionalized acceptors around one donor, nanoclusters free of large aggregates are formed. See FIGS. 6 and 10. An issue with current trends is the forming of crosslinking aggregates when multifunctionalized acceptors are used. FIG. 4 validates the monofunctionality in one embodiment of the capping conjugates. The inset in panel a of FIG. 4 is a schematic of an AuNP-dimer structure, having a single amino group for the subsequent steps. A preferred embodiment has one epitope for each capping conjugate in order for the optimal size and spacing leading to nanoclusters free of large aggregates. See FIG. 6.

However, other embodiments allow for detection of multiple epitopes at the same time. In one or more of these embodiments, the monofunctionalized capping conjugates are prepared in different groups, each group prepared as described with a single peptide epitope. In one or more of the multiple detection embodiments, the different groups are added sequentially to the core conjugates functionalized with the multiple Fabs with quenching measurement performed after each group is added. In one or more of these multiple detection embodiments, the different monofunctionalized capping conjugate groups are mixed together so that there is a cocktail of monofunctionalized capping conjugates comprising different single peptide epitopes. This cocktail can then be added to quench the core conjugates which are functionalized with multiple Fabs specific to the different groups of capping conjugates in the cocktail. In one or more of these embodiments, the capping conjugate cocktail can be exposed to the core conjugates functionalized with multiple Fabs in a sequence.

### Nanocluster Complex Assembly

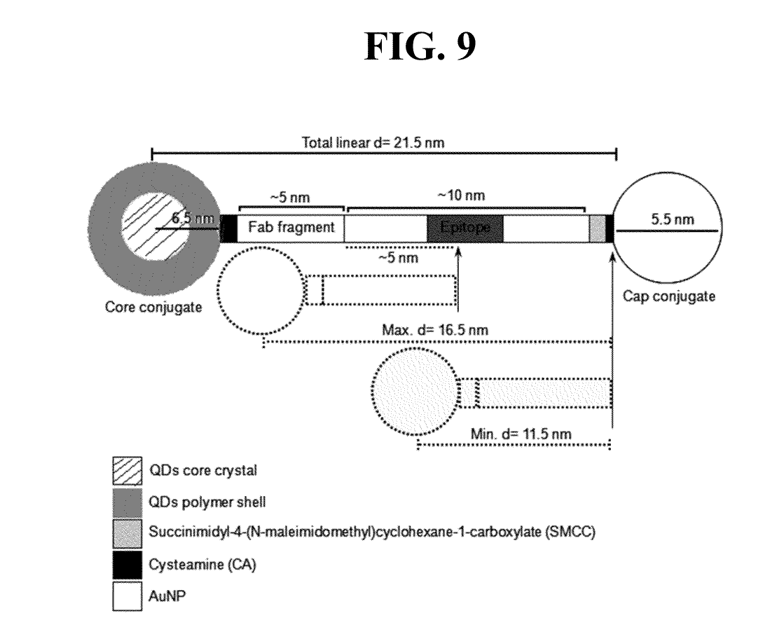

The cluster complex is assembled by combining the nanocluster capping conjugates and the single core nanoparticle conjugates. The capping and core conjugates self-assemble through the interaction between the two nanoconjugates by epitope-Fab interclustering elements. FIG. 9 shows the linear size of one embodiment of the nanocluster complex. The length of the nanocluster complex can be adjusted so as to create the optimal length for Complexes of different molar ratios of cap/core conjugates can be used ranging from greater than 0:1 to less than 10:1.

In a preferred embodiment, the metallic nanoparticles range in size from about 5-10 nm as to circumvent surface particle load limitation during cluster assembly. In a more preferred embodiment, metallic nanoparticles of about 5.5 nm in size are used. In a preferred embodiment, the peptide used is short so that combined with its monoclonal antibody Fab fragments a compact cap and core conjugates produce a theoretical interclustering distance of less than or equal to about 16.5 nm. See FIG. 9. This size is outside of the detection range of regular FRET, and much shorter than plasmonic resonator immunological schemes in the prior art, making it more advantageous than both FRET and previous NSET schemes.

As shown in FIG. 6, the quenching responses to increasing the Cap/Core ratio are shown and photoluminescence decreases with the higher Cap/Core ratio. Panel b of FIG. 6 shows the quenching efficiency with the increase in Cap/Core ratio while panel c shows the photoluminescence response to the same concentration of just core, core with caps but no epitope, and core with caps and epitope.

### Linkage

The QD-Fab linkage can occur through a number of ways including, but not limited to covalent bonds, ionic bonds, metallic bonds, hydrogen bonds, dipole-dipole moments, and van der Wall forces. A preferred embodiment comprises a quantum dot covalently linked to at least one Fab.

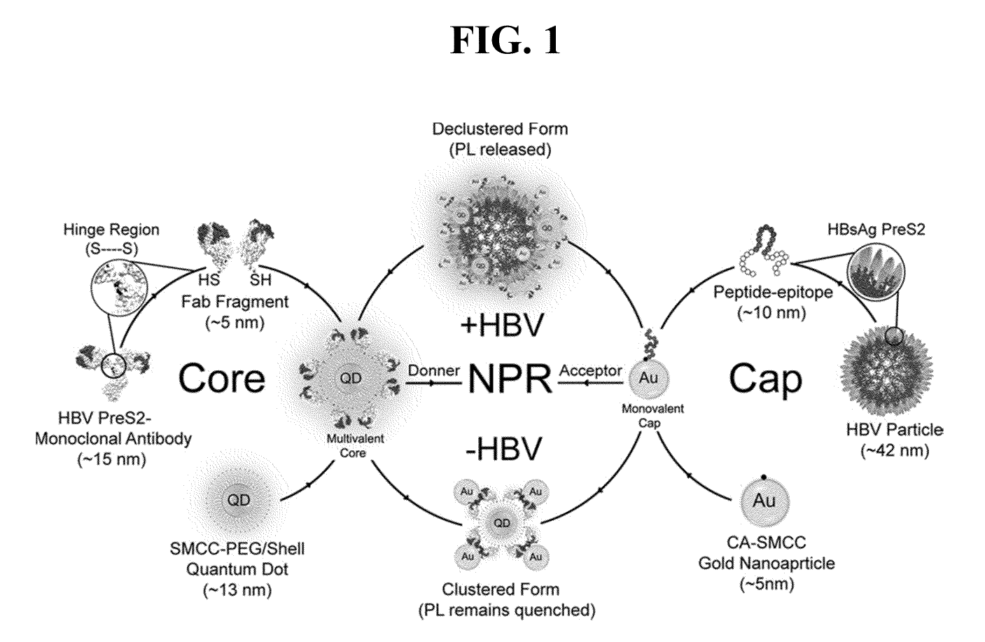

In a preferred embodiment, antibody-Fabs are relatively smaller antigen-binding fragments with monovalent structure targeting a short epitomic peptide. The Fab fragments are anchored on the Core surface via the thiol group pre-existing at the hinge region of digested antibody, so that the antigen recognition regions on Fab are oriented outward, and fully accessible to interact with acceptor capping conjugates or the competing epitope surface protein (right and central panel of FIG. 1). The orientation of Fabs provides rapid specific interaction as a result of reduced steric hindrance and reduced nonspecific binding, all issues commonly faced in large protein-antibody interactions.

### Method of Detection

First, a working solution made of capping conjugates (80 nM) and Core (20 nM) was prepared in borate buffer and incubated in darkness for 15 min at room temperature. For detection of viral antigens, 50 μL aliquots of HBsAg dilutions (0.0001, 0.001, 0.01, 0.05, 0.1, 0.5, 1, 5, 10 ng/mL) and HCVcoreAg dilutions (0.001, 0.01, 0.05, 0.1 ng/mL) were prepared in borate buffer. To the control experiment wells, only borate buffer was added. Using the same experimental set up, the cluster complex detection specificity was confirmed by exposure to different concentrations of a nonspecific protein, BSA (0.001, 0.01, 0.05, 0.1, 0.5, and 1 ng/mL) side by side to the same concentrations of HBsAg as a specific protein. The PL signal intensity of each well was collected after 15 min of incubation. The signal (background corrected) was plotted as the difference between the fluorescence recorded for each viral antigen (HBsAg and HCVcoreAg) concentration and that collected from the control.

For detection of viral particles, aliquots (50 μL) of the previously described working solution were mixed with equal volumes (50 μL) of different viral particle dilutions (1-1000 particle/μL in borate buffer) in the wells of a microtiter plate. After incubation for 15 min in room temperature, the fluorescence signal was collected. The signal (background corrected) was plotted as the difference between the fluorescence recorded for each virus concentration (HBV and HCV) and that collected from the control (no virus).

One or more embodiments utilize metallic nanoparticles and quantum dot nanoparticles with Fab antibody in the preassembled plasmonic resonance nanocluster, providing immunological detection of epitopes circumventing the issues large sizes impacting the distance-dependent resonance mechanism of nanomaterial surface energy transfer (NSET). This direct impact of NSET leads to poor plasmonic resonance yield and inefficient detection of microbes.

While several specific examples are described herein for plasmonic nanocluster complex detection of HBV, HCV, and EBV, the described nanoclusters can be used in detection of a variety of diseases. A person having skill in the art would know that in order to use the disclosed plasmonic nanocluster complex in other microbial and viral diseases, the Fab fragments should be customized to an antigen of a particular disease.

### EXAMPLE 1

### Detection of Hepatitis B Virus with Gold Nanoparticles

This embodiment is described in the journal article, “Hybrid Nanocluster Plasmonic Resonator for Immunological Detection of Hepatitis B Virus” published in ACS Nano, 2012 Vol. 6, No. 9, p 7634, and is incorporated herein in its entirety, including the Supporting Information material. As shown in FIG. 1, HBV NPR complex comprises two main conjugates of core conjugate (Core, left panel) and capping conjugate (Cap, right panel). The nanocluster core conjugates are produced as described below. Fab fragments were generated from monoclonal antibody against HBV preS2 antigen (cat no. ab8635, Abcam) using Mouse IgG1 Fab and F(a\acute{b})2 Micro Preparation Kit (cat no. 44680, Pierce).

For the preparation of Hepatitis B viral particles, HepG2-2.2.15 cells, a cell line that constitutively expresses replicating HBV from an integrated cDNA of the genome was used in this study for virus propagation and particle preparation. Cells were expanded and maintained in special Dulbecco's modified Eagle medium (DMEM, Gibco BRL), supplemented with 10% heat-inactivated Fetal bovine serum (FBS, Gibco BRL), 100 U/mL penicillin, and 100 mg/mL streptomycin, and cultured at 37° C. under a humidified atmosphere containing 5% CO2. The medium of the cells was changed every 2 days and the cells were washed by phosphate buffered saline (PBS, 1×, pH 7.4), trypsinized, and re-plated again until achieving 80-90% confluence maintained. Medium exposed to confluent cultures of HepG2-2.2.15 cells for 6 days was collected, and centrifuged at 10,000 rpm for 10 min at 4° C. to remove cellular debris. HBV particles were precipitated from medium samples with PEG 8000. The resulting pellet was resuspended in PBS (pH 7.4) and subjected to CsCl gradient (0.33 g/mL), then centrifuged at 53,000 rpm for 64 h in a MLA-80 rotor (Beckman Coulter, USA) at 4° C. The gradients were carefully divided into 250 fractions. The density of each fraction was determined by refractive index (WAY-2S Abbe refractometer, Shanghai Precision & Scientific Instrument Co., Shanghai, China). Fractions of densities between 1.2 and 1.3 g/mL which contain the complete virions were collected and dialyzed against PBS. HBsAg was confirmed using a microplate enzyme immunoassay kit, and virus concentration (genome equivalents) was determined by Real-time PCR using the commercial test from Roche Diagnostics.

The Fab fragments were generated from monoclonal antibodies against HBV preS2 (HBV preS2 mAb) by cleavage followed by reduction of its hinge region disulfide bond (cysteine residues), the target of proteolytic cleavage and reduction reaction. Peptide epitope corresponding in sequence to HBV preS2 antigen and specifically recognized by the Fabs was synthesized, and additionally modified with synthetic C-terminal cysteine (13-aa in length, HQTLQDPRVRGLC). 125 μL of IgG molecules (1 mg/mL) was first digested with immobilized ficin in the presence of 25 mM cysteine. Fabs were then separated from the intact Fc fragments and undigested IgG, and recovered by protein A column chromatography. The eluted Fab fractions were washed out by PBS and purified by Centricon (50 KDa MWCO, Millipore Carrigtwohill, Co.) centrifugation. Fractions containing the purified Fab fragments were pooled for future use, and the protein concentration was determined using the nanodrop technique.

The synthesized core conjugates were characterized by optical characterizations and gel electrophoresis technique (FIG. 5), and Fab fragments per core conjugate were quantified using bicinchoninic acid protein assay.

In this example of one embodiment, through the thiol-amine cross-linking chemistry using the SMCC cross-linker, the prepared Fabs and peptide epitope were directionally conjugated to quantum dots (QDs) and gold nanoparticles (AuNPs) to prepare multivalent Cores and monovalent Caps, respectively. Qdot 525 amine derivatized, PEG-coated nanocrystals were conjugated with the freshly prepared Fab fragments using Qdot 525 Antibody Conjugation Kit (cat no. Q22041MP, Invitrogen). The conjugation reaction was based on the efficient directional coupling of thiols that are present in reduced Fabs to the reactive maleimide groups present on the nanocrystals after SMCC activation. Because of the affinity interaction between Fab fragments and its specific peptide epitope, the caps and core conjugates assemble together resulting in AuNPs plasmonically resonance-quenching QDs emission. In the presence of HBV, the NPR complex is disassembled; the capping and core conjugates decluster, and PL signal reemerges to allow virus detection. In the absence of HBV, the NPR complex of caps-core conjugates remains clustered, allowing the plasmonic resonance of AuNP Caps to quench the photoluminescence (PL) of the QD Core. HBV capsid and antibody structures are modification and adaptation of data from the Protein Data Bank (HBV, 1QGT; antibody, 1IGT).

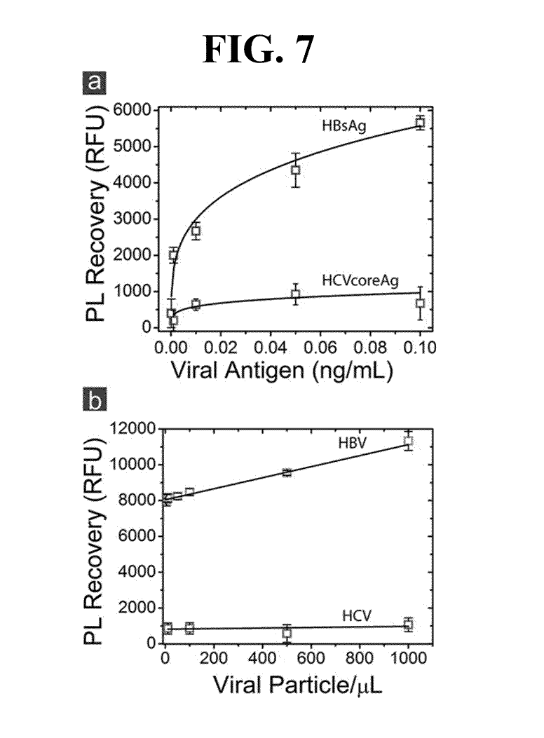

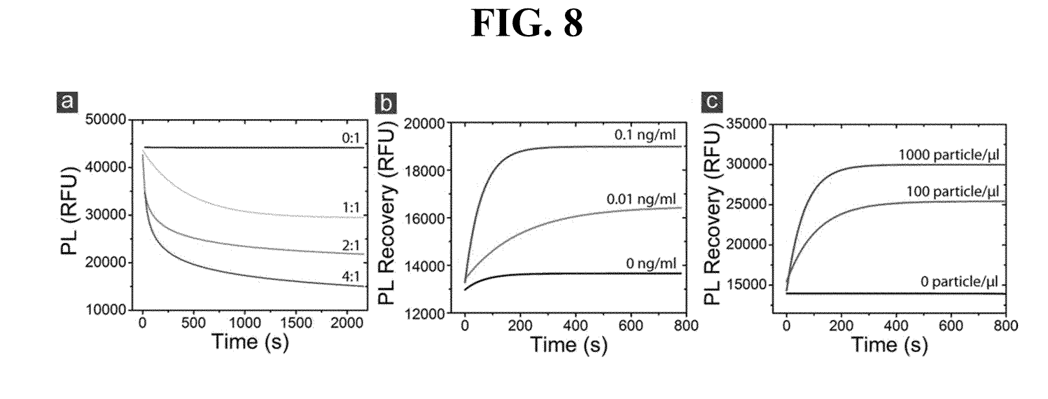

Results can be seen in FIG. 7 panel b where photoluminescence recovery signals increased with elevated virus concentration and no significant recovery signals recorded with HCV control particles. FIG. 8 panel a shows time-dependent variation of photoluminescence signal with respect to different caps/core ratios. Panels b and c of FIG. 8 represent the kinetics of the nanocluster complex disassembly caused by the addition of HBsAg and HBV concentrations, respectively. These results show a sensitive and specific nanocomplex for detection of HBV and its surface antigen HBsAg.

### EXAMPLE 2

### Detection of Hepatitis C Virus Using Gold Nanoparticles

HCV NPR complex comprises two main conjugates of core conjugate and capping conjugate. Fab fragments were generated from monoclonal antibodies against HCV E2 (HCV NS3 mAb) by cleavage. Peptide epitope corresponding in sequence to HCV NS3 antigen and specifically recognized by the Fabs was synthesized. Through the thiol-amine cross-linking chemistry using the SMCC cross-linker, the prepared Fabs and peptide epitope were directionally conjugated to quantum dots (QDs) and gold nanoparticles (AuNPs) to prepare multivalent cores and monovalent caps, respectively. Using AuNPs (5.5 nm) for the preparation of monofunctionalized capping conjugates by coupling with the targeted peptide epitope. Citrate-capped AuNPs were synthesized by the reduction of chloroaurate ions of chloroauric acid hydrated (AuCl₃.HCl.4H₂O, Au≧47.8%) by sodium borohydride (NaBH₄) in the presence of sodium citrate (C₆H₅Na₃O₇.2H₂O). Monomaleimide functionalized gold nanoparticles were prepared from the synthesized AuNPs by a facile scheme, which was based on a sequential ligand exchange reaction in the presence of stabilizing nonionic surfactant Tween-20. The surface of gold nanoparticles is first modified by the addition of cysteamine (CA) in a molar ratio of 1:1 to ensure one amino group for each particle, then 1-thioglycerol (TG) assemblea on the remaining surface area of the particles. Subsequently, the CA- and TG-stabilized gold nanoparticles were functionalized by SMCC, which interacts with the amino group (carried on CA) on the particle surface by its N-hydroxysuccinimide (NHS ester). A synthetic peptide epitope modified with C-terminal cysteine is synthesized. The peptide was allowed to couple through its terminal cysteine thiol group to the active maleimide group introduced to AuNPs surface by SMCC. The synthesized AuNPs particles and the prepared capping conjugates and their functional structure of peptide epitope were characterized by TEM, UV-vis, DLS, zeta potential, FT-IR, EDX, and Cy5 labeling absorption spectroscopy techniques.

Due to the affinity interaction between Fab fragments and its specific peptide epitope, the Caps (acceptor) and Core (donor) conjugates assemble together resulting in AuNPs plasmonically resonance-quenching QDs emission. In the presence of HCV, the NPR complex is disassembled; the capping and core conjugates decluster and PL signal reemerges to allow virus detection. In the absence of HCV, the NPR complex of caps-core conjugates remains clustered, allowing the plasmonic resonance of AuNP Caps to quench the photoluminescence (PL) of the QD Core.

### EXAMPLE 3

### Detection of Epstein-Barr Virus Using Gold Nanoparticles

Epstein-Barr virus (EBV) NPR complex comprises two main conjugates of core conjugate and capping conjugate. Fab fragments are generated from monoclonal antibodies against EBV membrane antigens MA-2, MA-4, MA-5, and MA-7 by cleavage. Peptide epitope corresponding in sequence to the EBV membrane antigens and specifically recognized by the Fabs is synthesized. Through the thiol-amine cross-linking chemistry using the SMCC cross-linker, the prepared Fabs and peptide epitope were directionally conjugated to quantum dots (QDs) and gold nanoparticles (AuNPs) to prepare multivalent Cores and monovalent Caps, respectively. Because of the affinity interaction between Fab fragments and its specific peptide epitope, the Caps (acceptor) and Core (donor) conjugates assemble together resulting in AuNPs plasmonically resonance-quenching QDs emission. In the presence of Eppstein Barr virus, the NPR complex is disassembled; the capping and core conjugates decluster and PL signal reemerges to allow virus detection. In the absence of Eppstein Barr virus, the NPR complex of caps-core conjugates remains clustered, allowing the plasmonic resonance of AuNP Caps to quench the photoluminescence (PL) of the QD Core.

Having described the basic concept of the invention, it will be apparent to those skilled in the art that the foregoing detailed disclosure is intended to be presented by way of example. Various alterations, improvements, and modifications are intended to be suggested and are within the scope and spirit of the present invention. Additionally, the recited order of the elements or sequences, or the use of numbers, letters or other designations therefore, is not intended to limit the claimed processes to any order except as may be specified. All ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art, all language such as up to, at least, greater than, less than, and the like refer to ranges which can be subsequently broken down into sub-ranges as discussed above. Accordingly, the invention is limited only by the following claims and equivalents thereto. All publications and patent documents cited in this application are incorporated by reference in their entirety for all purposes to the same extent as if each individual publication or patent document were so individually denoted.

We claim:

1. A nanocluster complex for identifying the presence of one or more microbes comprising:
a. a single core nanoparticle conjugate (CNC), wherein said CNC comprises a quantum dot covalently linked to one or more fragment antigen-binding (Fab) antibodies;
b. one or more multiple capping nanoparticle conjugates (MCNCs), wherein each said MCNC comprises a metal nanoparticle functionalized with one or more peptide epitopes; and
c. at least one linkage between said CNC and one or more said MCNCs, wherein said linkage comprises one or more peptide epitopes of said MCNCs and said Fab antibodies targeting said epitopes on said CNC.

2. The complex of claim 1 wherein said linkage comprises one peptide epitope and said Fab antibody targets said epitope.

3. The complex of claim 2 wherein:
a. said single peptide epitope comprises at least 85 percent homology to the sequence of Hepatitis B Virus surface antigen preS2;
b. said Fab fragments targeting said epitope are derived from monoclonal antibody against said HBV preS2 surface antigen; and
c. said HBV surface antigen preS2 and said Fab antibody against said HBV preS2 surface antigen mediate the preassembly of said cap-core nanocluster complex.

4. The complex of claim 1, wherein said MCNCs comprise a metal selected from the group consisting of elemental metals and metal salts.

5. The complex of claim 3 wherein said CNC and said MCNCs when mixed, assemble together with well-controlled spatial distance, orientation, and molar ratio.

6. The complex of claim 3 wherein said CNC comprises Fab fragments covalently linked to amine-derivatized, polyethylene glycol-coated quantum dots.

7. The complex of claim 4 wherein said MCNCs comprise a metal selected from the group consisting of gold, copper, silver, nickel, palladium, platinum, cobalt manganese, titianium, vanadium, chromium, zinc, iron, selenium, and the oxides, hydroxides, sulfides, selenides, and tellurides of the foregoing and combinations thereof.

8. The complex of claim 7 wherein said metal of said MCNCs is gold.

9. The complex of claim 8 wherein each said MCNC is monofunctionalized with a single peptide epitope.

10. The complex of claim 8 wherein said gold capping conjugate nanoparticles are spherical in shape with an average diameter between about 4 and 7 nm.

11. The complex of claim 8 wherein said gold capping conjugate nanoparticles are spherical in shape with an average diameter between about 4.128 and about 6.882 nm.

12. The complex of claim 9 wherein multiple said MCNCs in proximity to one said CNC will produce discrete nanoclusters, free from large aggregates.

13. The complex of claim 1, wherein said linkage comprises more than one peptide epitope and said fragment antigen-binding antibodies target said epitopes.

14. The complex of claim 2 wherein:
a. said single peptide epitope comprises at least 85 percent homology to the sequence of Hepatitis C Virus E2 epitope;
b. said Fab fragments targeting said epitope are derived from monoclonal antibody against said HCV E2 epitope; and
c. said HCV E2 epitope and said Fab antibody against said HCV E2 epitope mediate the preassembly of said cap-core nanocluster complex.

15. The complex of claim 2 wherein:
a. said single peptide epitope comprises at least 85 percent homology to the sequence of Epstein-Barr virus membrane antigens
b. said Fab fragments targeting said epitope are derived from monoclonal antibody against said EBV membrane antigens; and
c. said EBV membrane antigens and said Fab antibody against said EBV membrane antigens mediate the preassembly of said cap-core nanocluster complex.

16. The complex of claim 15 wherein said Epstein-Barr virus membrane antigens are selected from the group of: EBV MA-2, EBV MA-4, EBV MA-5, and EBV MA-7.
